SEQUENCE LISTING (5 sequences)

SEQ ID NO: 1 - DNA, 18 bp, Artificial Sequence
  FISH probe
cttgagaatttcttcccc

SEQ ID NO: 2 - DNA, 22 bp, Artificial Sequence
  FISH probe
caccgcgacatgctgattcgcg

SEQ ID NO: 3 - DNA, 19 bp, Artificial Sequence
  FISH probe
gcaatacattcagtggcga

SEQ ID NO: 4 - DNA, 21 bp, Artificial Sequence
  FISH probe
gttcgccactgaatgtattgc

SEQ ID NO: 5 - DNA, 18 bp, Artificial Sequence
  FISH probe
attagatgccgactcggg

(19) United States
(12) Patent Application Publication
(10) Pub. No.: US 2022/0056505 A1
(43) Pub. Date: Feb. 24, 2022

(54) Methods for Detecting Cell Wall-Deficient Bacteria (CWDB) in Biological Samples

(71) Applicant: Biotrack Holding B.V., Sneek (NL)

(72) Inventor: Gijsbert Johan Jansen, Giekerk (NL)

(73) Assignee: Biotrack Holding B.V., Sneek (NL)

(21) Appl. No.: 17/299,885

(22) PCT Filed: Dec. 5, 2019
(86) PCT No.: PCT/NL2019/050808

§ 371 Date: Jun. 4, 2021

(30) Foreign Application Priority Data
Dec. 6, 2018 (NL) 2022147

(51) Int. Cl. C12Q 1/689; C12Q 1/6841; G01N 21/64

(52) U.S. Cl. CPC C12Q 1/689; G01N 2021/6439; G01N 21/6428; C12Q 1/6841

(57) ABSTRACT

The invention relates to the field of microbiology, in particular to diagnosing cell wall deficient/defective bacteria (CWDB) in clinical samples. Provided is a method for determining the presence of CWDB in cells, comprising the steps of (i) subjecting a biological sample comprising cells to be analyzed for the presence of CWDB to fluorescence in-situ hybridization (FISH) analysis using a fluorescently labeled nucleic acid probe capable of specifically hybridizing with a target nucleic acid sequence of the predetermined bacterial target, (ii) collecting optical images of a plurality of cells, preferably using fluorescence-based detection fitted with the appropriate optical filter, to detect the fluorescently labeled nucleic acid probe, and digitally storing optical images; (iii) binarizing the digital optical images to discriminate fluorescent objects from an indifferent background; (iv) subjecting selected fluorescent objects on the binary image to a morphometric analysis comprising determining for a plurality of fluorescent objects at least the surface area and the roundness; and (v) correlating the results of the morphometric analysis with the presence of CWDB, wherein fluorescent objects having a surface area in the range of 5-50 μm² and a roundness in the range of 0.800 to 1.000 are indicative of CWDB.

### RELATED APPLICATIONS

This application is a National Stage Application under 35 U.S.C. 371 of co-pending PCT application PCT/NL2019/050808 designating the United States and filed Dec. 5, 2019; which claims the benefit of NL application number 2022147 and filed Dec. 6, 2018, each of which are hereby incorporated by reference in their entireties.

The invention relates to the field of microbiology, in particular to diagnosing cell wall deficient/defective bacteria in biological samples, like blood cells. Some pathogenic bacteria (e.g. Coxiella burnetii, Borrelia burgdorferi and Chlamydia trachomatis) can exist as intracellular organisms that can revert to wildtype bacteria outside a host cell. In the intracellular phase, these pathogens do not have a cell wall (hence the term “cell wall-deficient/defective bacteria (CWDB) or L-form bacteria). They typically evade the host's immune system by hiding in white blood cells, including lymphocytes, monocytes and macrophages. Thus, removal of the bacterial cell wall components may contribute to an evasion from the host defense system and the intracellular survival of the bacteria which may ensure their long-term persistence. Whereas CWDB usually do not trigger the classical inflammatory responses, they can persist for prolonged periods, e.g. up to several decade, in the host organism and cause a variety of vague symptoms.

The pathogenic potential of CWDB for humans and (laboratory) animals has been the subject of many journal reports and books. Whereas the results of these studies have often been inconclusive and sometimes contradictory, other studies seem to provide experimental and clinical evidence supporting the concept that CWDB may serve as cryptic agents of disease in a variety of human infections. See for example Onwuamaegu et al. (J. Int. Medical research 33(1):1-20, February 2005) and Astrauskiene et al. (Clin Exp Rheumatol. 2007 May-June; 25(3):470-9).

The controversy around the role of CWDB in (human) disease is partly related to technical difficulties that are encountered when testing for the presence of CWDB in a sample.

Conventional procedures used in clinical microbiology cannot be used, since CWDB do not grow in the usual media and PCR-based methods cannot discriminate between the extracellular and intracellular form. Therefore, special methods should be applied. Serology methods involving detection of e.g. C. burnetii antibodies are currently accepted as the “gold standard” since it can provide a direct insight into functioning of patients' immune system. However, intra-leukocyte C. burnetii do not give rise to a humoral immune response and can thus lead to false-negative results. CWDB can be determined by a variety of other techniques, such as electron microscopy, DNA detection, RNA detection, in situ hybridization and the presence of specific proteins. However, it appears that the methods involved in these newer techniques are often difficult to reproduce and many (clinical) centers do not accept their validity.

Accordingly, the present inventors realized the need for an improved method for detecting CWDB which (i) identifies bacteria to the genus and/or species level; (ii) discriminates between intracellular and extracellular variants and (iii) distinguishes CWDB from native cells. Ideally, the novel method should be highly reliable and allow for standardization e.g. in a clinical setting. Surprisingly, it was found that at least some of these goals could be met by the provision of a method wherein FISH analysis is combined with a unique approach wherein “true” CWDB cells are identified on the basis of morphometric parameters.

In one embodiment, the invention provides a method for determining the presence of predetermined cell wall-deficient bacteria (CWDB) in cells, comprising the steps of
(i) subjecting a biological sample comprising cells to be analyzed for the presence of CWDB to fluorescence in-situ hybridization (FISH) analysis using a fluorescently labeled nucleic acid probe capable of specifically hybridizing with a target nucleic acid sequence of the predetermined bacterial target;
(ii) collecting optical images of a plurality of cells, preferably using fluorescence-based detection fitted with the appropriate optical filter for detecting the fluorescently labeled nucleic acid probe, and digitally storing optical images;
(iii) binarizing the digital optical images and discriminating fluorescent objects from an indifferent background;
(iv) subjecting selected fluorescent objects on the binary image to a morphometric analysis comprising determining for a plurality of objects at least the surface area and the roundness; and
(v) correlating the results of the morphometric analysis with the presence of CWDB, wherein fluorescent objects having a surface area in the range of 5-50 μm² and a roundness in the range of 0.800 to 1.000 are indicative of CWDB.

The present invention can be applied to any type of biological sample (including tissue specimens) comprising host cells to be analyzed for the presence of CWDB. For example, the sample comprises blood cells, renal cells, in particular those forming renal pelvis wall, epithelial cells, e.g. of the bladder, neuronal cells, endocardial cells, myocardial cells, hepatocytes, and any combination thereof.

In one aspect, the sample comprises blood cells, preferably white bloods, lymphocytes, monocytes and/or macrophages. Preferably, the biological sample is a clinical sample. For example, the method is readily performed on a peripheral blood sample, venous blood sample or capillary blood sample. In a specific aspect, the sample is a blood sample obtained from a blood donor (donated blood sample). Hence, also provided is a method for the screening of donates blood samples for the presence of CWDB, comprising steps (i) through (iv) as defined herein above. This allows for the (automated) screening of all blood donations for evidence of CWDB infection prior to the release of the blood and its components for clinical and/or manufacturing use, e.g. prior to blood transfusion.

In another embodiment, the biological sample is a tissue sample. A method of the invention finds its use in diagnosing any mammalian species, and any veterinary use is also envisaged. However, human samples are particularly preferred.

In step (i) of a method provided herein, a biological sample comprising cells to be analyzed for the presence of CWDB is subjected to fluorescence in-situ hybridization (FISH) analysis using a fluorescently labeled nucleic acid probe capable of specifically hybridizing with a target nucleic acid sequence of the predetermined bacteria. FISH is a molecular cytogenetic technique that uses fluorescent probes that bind to only those parts of the 16S ribosomal RNA with a 100% degree of sequence complementarity. It was developed by biomedical researchers in the early 1980s, and is used to detect and localize the presence or absence of specific DNA sequences in micro-organisms. FISH-mediated detection of CWDB is also known in the art. For example, Melenotte et al. (Blood, 2015 Oct. 13, pp. 113-121) relates to detection of Coxiella burnetii in infected cells using FISH analysis, and discloses fluorescently labeled FISH probes for diagnosis and quantitive analysis of C. burnetii infection. Poppert et al. (Appl. Envir. Microbiology 2002-08-01, pp. 4081-4089) concerns detection and differentation of Chlamydiae by FISH.

Fluorescence microscopy can be used to find out where the fluorescent probe is bound to the chromosomes. FISH is often used for finding specific features in DNA for use in genetic counseling, medicine, and species identification.

A person skilled in the art will understand and appreciate that the concept underlying the invention is applicable to any CWDB (or L-form bacteria) that are known to date or that are still to be discovered. The term “predetermined” reflects the fact that, in a method of the invention, a sample is diagnosed for one or more CWDB of interest by the use of one or more fluorescently labeled nucleic acid probe(s) capable of specifically hybridizing with a target nucleic acid sequence of the predetermined bacterial target organism(s). In one embodiment, the predetermined CWDB is known or suspected to be related to a disease or disorder. For example, the CWDB is selected from the group consisting of the genus Borrelia, Bacillus, Salmonella, Brucella, Mycobacterium, Coxiella and Chlamydia. In one embodiment, it is selected from Salmonella typhi, S. paratyphi A, S. paratypi B and Brucella spp. In another embodiment, the CWDB is Coxiella burnetii, the bacterium causing Q-fever.

In a method provided herein, the cells contained within the biological sample are typically immobilized onto a solid support, followed by contacting the cells with a fixation and/or permeabilisation reagent according to known procedures, e.g. those used in conventional FISH analysis. Preferably, the solid support is a glass or polycarbonate slide. In a specific aspect, it is a microscopic glass slide.

The target nucleic acid sequence can be DNA, chromosomal DNA, RNA, messenger RNA (mRNA) or ribosomal RNA. It is also possible to stain for two or more distinct predetermined CWDB by using multiple bacterium-specific probes, each probe carrying a distinct fluorescent label. Hence, a method of the invention also encompasses performing multiplex CWDB-FISH analysis.

Step (ii) comprises collecting optical images of a plurality of cells, preferably using fluorescence based detection using the appropriate optical filter, to detect the fluorescently labeled nucleic acid probe, and digitally storing optical images. For example, photographic images are taken of at least 50 white blood cells under UV exposure. Images are stored digitally, preferably in TIFF, JPEG or BMP format. In one embodiment, step (ii) comprises the combination of fluorescence microscopy and a charge coupled device

In step (iii), digital optical images are binarized to identify and select fluorescent objects. More in particular, the digital optical images are binarized to discriminate fluorescent objects from an indifferent background. It is well known in the art that, in an effort to standardize results, digital images of fluorescent labelled organisms or structures can be processed by (semi-)automated image analysis software packages. In this automatization process, a greyscale image is converted into a binary (two possible pixel values, normally black and white) image. This conversion process is called thresholding. Binarization procedures are known per se in the art. See Gonzalez et al. (2002). Thresholding. In Digital Image Processing, pp. 595-611. Pearson Education. ISBN 81-7808-629-8; Luessi et al. J. of Electronic Imaging, vol. 18, pp. 013004+, 2009. doi:10.1117/1.3073891; Lai et al. Efficient Circular Thresholding, IEEE Trans. on Image Processing 23(3), pp. 992-1001 (2014). doi:10.1109/TIP.2013.2297014; and Umbaugh (2018). Digital Image Processing and Analysis, pp 93-96. CRC Press. ISBN 978-1-4987-6602-9

In a preferred embodiment, it comprises the use of an algorithm which implements biologically significant variables (e.g., area or/and size of a typical bacterial cell), such as the method described in the study by Tamminga et al. (2016), which is herein incorporated by reference. Thereafter, in step (iv), selected fluorescent objects on the binary image are subjected to a morphometric analysis comprising determining for a plurality of objects at least the surface area and the roundness. This analysis may be performed manually using conventional image-analysis software, such as ImageJ or MIPAR. Alternatively, step (iv) is performed in an automated fashion employing an appropriate software module. Very good results can be obtained with a software module of BioTrack-MED system.

Hence, per each digitized CWDB the surface area (expressed in μm2 based on number of pixels per object) and the roundness per object is determined.

The surface area of an object is determined by the number of pixels in the cell. Pixels are picture elements of the digitized image and have units of area, referenced to the image not the sample. Each pixel has dimensions defined as fractions of the frame height and width, and has a spot intensity. Pixel sizes depend on the microscope magnification, the size of the imaging chip in the camera, and the number of pixels in the resulting image.

The roundness of an object/cell is suitably calculated according to the prescribed method of the National Institute For Standards in Technology, NIST. https://www.itl.nist.gov/div898/handbook/mpc/section3/mpc344.htm. The roundness parameter will be 1.000 for perfectly round objects, and will be lower to the degree that the object is elongated. Since this measurement is less sensitive to local irregularities in the perimeter, a more-or-less circular object with a wobbly perimeter will also be close to 1.000 with respect to roundness. Then, based on the parameter scores obtained per object in step (iv) of a method of the invention, individual objects are selected in step (v) according to the following inclusion criteria for CWDB: 5 μm²<Surface Area<50 μm² and 0.800<Roundness<1.000. Objects which meet these criteria are deemed to be CWDB. The fact that they fluoresce (and thus are positively marked with a target-specific nucleic DNA probe) also determines the identity of the CWDB.

Therefore, step (v) comprises correlating the results of the morphometric analysis with the presence of CWDB, wherein fluorescent objects having a surface area in the range of 5-50 μm² and a roundness in the range of 0.800 to 1.000 are indicative of CWDB.

A method of the invention may suitably comprise one or more further steps. In one embodiment, it includes providing a clinical diagnosis by determining the relative abundance of CWDB-positive (blood) cells in the sample, and correlating the relative abundance with the presence of a CWDB-related disease condition. It was found that analyzing only 50 white blood cells is sufficient for obtaining a clinically significant test outcome. Therefore, in one aspect a method of the invention comprises analyzing at least 50 white blood cells. More in particular, it was demonstrated that an amount of at least 3 CWDB-positive blood cells per 50 white blood cells is indicative of a CWDB-related disease condition.

As is shown herein below, very good results were obtained when staining blood cells for Coxiella burnetii, the bacterium that causes the disease Q which is commonly found in sheep, goats and cattle. The bacterium can also infect pets, including cats, dogs and rabbits. These animals transmit the bacteria through their urine, feces, milk and birthing products—such as the placenta and amniotic fluid. When these substances dry, the bacteria in them become part of the barnyard dust that floats in the air. The infection is usually transmitted to humans through their lungs, when they inhale contaminated barnyard dust. A Q fever recurrence can give rise to serious complications, including endocarditis, lung issues, pregnancy problems, liver damage and meningitis. Up to now, Q-fever diagnosis and follow-up is usually based on serology (looking for an antibody IgG/IgM response) rather than looking for the organism itself. Whereas molecular detection of bacterial DNA is increasingly used, culturing is technically difficult and not routinely available in most microbiology laboratories.

The present invention now provides a robust and reproducible method for determining the presence of Coxiella burnetii in cells, comprising the steps of (i) subjecting a biological sample comprising cells to be analyzed for the presence of C. burnetii to fluorescence in-situ hybridization (FISH) analysis using a fluorescently labeled nucleic acid probe capable of specifically hybridizing with a target nucleic acid sequence of the predetermined bacteria; (ii) collecting optical images of a plurality of cells using fluorescence-based detection using the appropriate optical filter to detect the fluorescently labeled nucleic acid probe, and digitally storing optical images; (iii) binarizing the digital optical images to identify and select fluorescent objects; (iv) subjecting selected fluorescent objects on the binary image to a morphometric analysis comprising determining for a plurality of objects at least the surface area and the roundness; (v) correlating the results of the morphometric analysis with the presence of C. burnetii, wherein fluorescent objects having a surface area in the range of 5-50 μm² and a roundness in the range of 0.800 to 1.000 are indicative of C. burnetii-positive cell. Exemplary C. burnetii-specific FISH probes for use in a method of the invention include (5′-CTTGAGAATTTCTTCCCC-3′) and (5′-CACCGCGACATGCTGATTCGCG-3′), which specifically target the C. burnetii 16S rRNA sequences (Melenotte et al. Blood 2016 127:113-121). Importantly, it was observed that an amount of at least 3 Coxiella burnetii-positive white blood cells per 50 white blood cells provides a statistically significant indicator of Q-fever in a human subject.

In another embodiment, the invention relates to detecting Borrelia burgdorferi CWDB. B. burgdorferi is the causative agent of Lyme's disease, which affects an estimated 300,000 people annually in the United States. When treated early, the disease usually resolves, but when left untreated, it may result in symptoms such as arthritis and encephalopathy. Current Lyme's disease tests rely solely on serology and measure Borrelia antibodies in the blood, or in the cerebrospinal fluid (CSF) if there are clinical symptoms of central nervous system disease. Despite the presence of new and improved assays, current laboratory diagnosis still requires optimization. The variety of species and their different geographic distributions are the reasons why standards and recommendations are not the same for all of the main geographic regions, ie, USA, Asia, and Europe. See the review by Zajkowska (Dove Press; Volume 2014:4 Pages 33-44). The present invention provides a new, antibody-independent approach allowing for the detection and quantification of B. burgdorferi-positive white blood cells. Exemplary FISH probes for use in a method of the invention include those reported by Hammer et al. (Microbiology (2001), 147, 1425-1436. In a preferred embodiment, at least one of the oligonucleotide probes 5′-GCAATACATTCAGTGGCGA-3′ and 5′-GTTCGCCACTGAATGTATTGC-3′ is used.

In one embodiment, the method further comprises providing a clinical diagnosis of Lyme's disease by determining the relative abundance of B. burgdorferi-positive white blood cells in the sample, and correlating the relative abundance with the presence of Lyme's disease. In one aspect, it comprises analyzing at least 50 white blood cells and providing a positive Lyme's disease diagnosis if at least 3 B. burgdorferi-positive white blood cells per 50 white blood cells are detected.

In a still further embodiment, the invention relates to detecting Chlamydia CWDB. The genus Chlamydia comprises amongst others the species Chlamydia pneumoniae, Chlamydia trachomatis, Chlamydia psittaci, and Chlamydia pecorum. Chlamydiae are characterized by a complicated, obligate intracellular developmental cycle which is unique among the prokaryotes. The metabolically active, obligate intracellular chlamydial reticulate bodies multiply within their eukaryotic host cells and subsequently differentiate to inactive elementary bodies, which are released and are able to infect new host cells to start a new developmental cycle.

Chlamydiae cause a wide variety of diseases in humans and animals, including infections of the eye and the respiratory and the genital tracts. More recently, they were also associated with cardiovascular disease, atherosclerosis, and intrinsic asthma. Diagnosis of chlamydial infections can be attempted by various detection methods, including culture, serology and nucleic acid amplification. However, in spite of the multiplicity of available test methods, the detection of Chlamydiae from clinical specimens remains a major challenge for routine laboratories. Especially, laboratory diagnosis of the fastidious C. pneumoniae is currently hampered by a lack of standardized and validated assays leading to a considerable interlaboratory variance of test results. This is one of the major reasons why the role of C. pneumoniae in respiratory tract disease as well as in atherosclerosis is still unclear to this day. Therefore, both standardizing of available test assays and development of new molecular methods are urgently needed to obtain reliable tools for sensitive and specific detection of Chlamydia spp.

Suitable Chlamydia-specific FISH probes for use in a method of the invention include those shown in Table 1 of Poppert et al. (Appl Environ Microbiol. 2002 August; 68(8): 4081-4089). For example, the C. trachomatis-specific oligonucleotide 5′-ATTAGATGCCGACTCGGG-3′ is used.

### LEGENDS TO THE FIGURES

FIG. 1: Schematic representation of the different steps of an exemplary method of the invention. Panel A depicts a blood sample that is not stained, phase-contrast image, (1000× magn). In step 1, the blood sample is hybridized with a C. burnetii-specific fluorescently labeled probe. Panel B depicts a fluorescence image (1000× magn.) showing fluorescent objects representing C. burnetii cells. In step 2, the fluorescent image is binarized, for example according to Tamminga et al (2016) to yield a binary image (panel C). In step 3, extracellular cells of C. burnetii are deleted from the image on the basis of roundness (rod vs. circle) and aggregates of intracellular cells of C. burnetii are discarded on the basis of surface area>50 um², to yield a binary image only showing “true” CWDB cells (panel D).

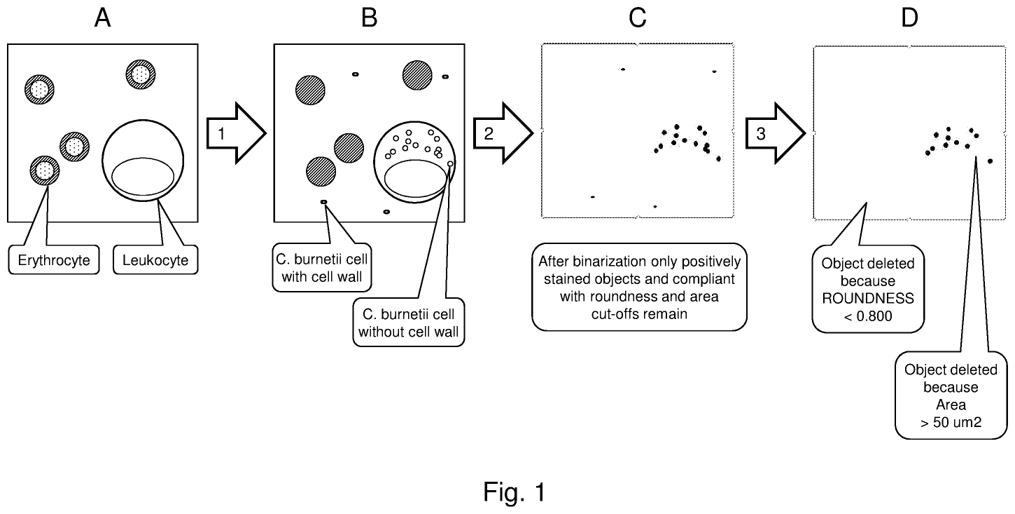

FIG. 2: Phase contrast (left hand panels) and fluorescence micrographs (right hand panels) of whole blood samples of two Q-fever patients (top and bottom panels) samples in situ hybridized with Coxiella burnetii-specific probe. 400× magnification.

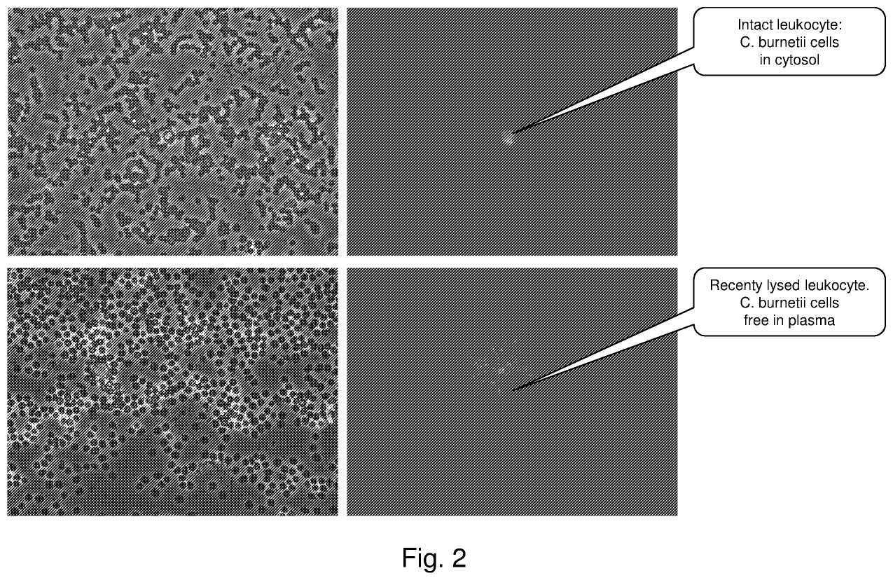

### EXPERIMENTAL SECTION

Materials
1 vacutainer of venous, heparinized blood of up to 8 hours old (if kept at 4° C.)
2 object slides
1 coverslip
Fluorescence microscope with CCD camera and software for acquisition of image files (JPEG, BMP or TIFF)
10 ng/μ1 fluorescently labeled, single strain DNA probe specific for CWDB under test in standard hybridization buffer ((0.9 M NaCl, 20 mm Tris HCl, pH 7.5 1 m/v % SDS, 25 m/v % Triton-X)
Standard washing-buffer (0.9 M NaCl, 20 mm Tris-HCl pH 7.5)
V/V % methanol (70%)
Ultra-pure water
Incubator (50° C.)

Methods

Sample Preparation
100μ of whole blood is mixed with 100 μl of ultrapure sterile water and incubated at room temperature during 10 seconds
After 10 seconds a further 100 μl of ultra-pure water (2.7 m/v (%) NaCl) is added to the lysate to establish isotonicity.
The mix is then centrifuged during 10 min at 2000 rpm
Supernatant is discarded
Pellet consists of purified and concentrated white blood cells (WBC)

Slide Manufacturing

Pipette 5 μl of WBC Pellet on an Object Glass
Streak out the WBC pellet over the glass surface with a second object glass at an angle of about 30 degrees.
Dry the object glass to the air during 15 minutes

Fluorescence In Situ Hybridization
Fix the dried object glass during 15 minutes in 70% v/v methanol
Dry the object glass to air during 15 minutes
pipette 200 μl oligonucleotide probe-mix (10 ng/μ1, 5′-end Cye-labelled oligonucleotide CACCGCGACATGCTGATTCGCG in hybridization buffer) on the object glass and cover with coverslip
Place the covered object glass in a petri dish in which a wet piece of paper is deposited previously
Place the Petri dish in an incubator at 50° C.
Hybridize during 1 hour
Place covered object glass in 50 ml Falcon tube filled with wash buffer
Wash at 50° C. during 30 minutes
Take object glass from Falcon tube (the coverslip is soaked off and stays behind)
Dry to air during 15 min
pipette 20 μl VectaShield™ or any other embedding medium on the object glass and cover with a new cover slip
Fix the corners of the coverslip to the object glass using nail polish.

Image Acquisition
Adjust the fluorescence microscope to a magnification of 400× and choose the excitation filter which is appropriate for fluorescent Cy3 label
Under UV exposure, take photograph images of at least 50 white blood cells and store the images digitally in TIFF, JPEG or BMP format.

This completes the laboratory-part of this method. The end result is a set of digital photos in which, in total, 50 white blood cells (whether or not fluorescent) are displayed. Below follows the algorithm for subsequent morphometric analysis.

Morphometric Analysis

The morphometric analysis is performed on each individual digital image and consists of the following steps:
Fluorescent objects are identified and selected using the recent image segmentation algorithm of Tamminga et al. (J. Microbiol. Meth. (2016) September, 128, 118-124)) to yield a binary image containing only 1 or 0 values.
The following parameters are calculated per each digitized CWDB (1) surface area (expressed in μm2 based on number of pixels per object) and (2) the roundness per object (according to the prescribed method of the National Institute For Standards in Technology, NIST, (https://www.itl.nist.gov/div898/handbook/mpc/section3/mpc344.htm)
Then (based on the parameter scores per object) individual objects are selected according to the following inclusion criteria: 5 μm2<Surface Area<50 μm2 and 0.800<Roundness<1.000
Objects which meet these criteria are deemed to be CWDB. The fact that they fluoresce (and thus are positively marked with a species specific DNA probe) also determines the identity of the CWDB.
If 3 or more of the 50 white blood cells in a sample are found to be positive (i.e. comprising CWDB), the blood sample as clinically diagnosed as being positive for (chronic) Q fever, or Q fever associated fatigue syndrome.

The above morphometric analysis can be performed either manually (using existing image analysis software as ImageJ or MIPAR) or fully automated through e.g. the software modules of the BioTrack-MED™ system.

The methodology described here allows for simultaneous measurement of the identity, the phase (intra-or extracellular) and the status (CWDB or native). On the next page an overview of the results achieved so far is presented.

The method described in the previous section was subsequently applied to a number of blood samples. Blood samples were obtained from 10 patients diagnosed according to clinical applicable standard for Q-fever and from 10 healthy volunteers originating from a Dutch general practitioners office. The ages and sexes of the blood donors were unknown. The samples were all anonymized and consisted of at least 4 ml heparinized blood in plastic vacutainer.

On each of these 20 anonymous samples is performed above and the results of this research are presented in Table 1.

TABLE 1
WBS-scores in Q-fever patients and healthy volunteers

| | | # healthy | No. positive |
|---|---|---|---|
| # Q-fever patient | No. positive WBC* | volunteer* | WBC* |
| 1 | 9 | 1 | 0 |
| 2 | 5 | 2 | 1 |
| 3 | 8 | 3 | 1 |
| 4 | 6 | 4 | 0 |
| 5 | 7 | 5 | 2 |
| 6 | 7 | 6 | 1 |
| 7 | 11 | 7 | 0 |
| 8 | 9 | 8 | 0 |
| 9 | 13 | 9 | 1 |
| 10 | 9 | 10 | 0 |
| Total | 84 | | 6 |
| Mean | 8.4 | | 0.6 |

The number of WBC that stained positive for cell wall deficient C. burnetii in a total of 50 WBC was determined.

From these results it can be concluded (and endorsed by student's T-test) that the numbers of CWDB-positive white blood cells in Q-fever patients are significantly higher than in healthy volunteers. This demonstrates that the combination of FISH and Morphometry provides a valuable diagnostic tool for the detection of CWDB's.

1. A method for determining the presence of predetermined cell wall-deficient bacteria (CWDB) in cells, comprising the steps of
(i) subjecting a biological sample comprising cells to be analyzed for the presence of CWDB to fluorescence in-situ hybridization (FISH) analysis using a fluorescently labeled nucleic acid probe capable of specifically hybridizing with a target nucleic acid sequence of the predetermined bacterial target,
(ii) collecting optical images of a plurality of cells, and digitally storing optical images
(iii) binarizing the digital optical images and discriminating fluorescent objects from an indifferent background
(iv) subjecting selected fluorescent objects on the binary image to a morphometric analysis, said morphometric analysis comprising determining for a plurality of fluorescent objects at least the surface area and the roundness; and
(v) correlating the results of the morphometric analysis with the presence of CWDB, wherein fluorescent objects having a surface area in the range of 5-50 μm² and a roundness in the range of 0.800 to 1.000 are indicative of CWDB.

2. The method according to claim 1, wherein the sample comprises white blood cells, lymphocytes, monocytes and/or macrophages.

3. The method according to claim 1, wherein the biological sample is a blood sample.

4. The method according to claim 1, wherein the predetermined CWDB are disease-related CWDB.

5. The method according to claim 4, wherein the CWDB is selected from the group consisting of the genus Borrelia, Bacillus, Salmonella, Brucella, Mycobacterium, Coxiella and Chlamydia.

6. The method according to claim 5, wherein the CWDB is Coxiella burnetii, Borrelia burgdorferi, Chlamydia pneumoniae, Chlamydia trachomatis, Chlamydia psittaci or Chlamydia pecorum.

7. The method according to claim 1, wherein the target nucleic acid sequence is DNA, chromosomal DNA, RNA, messenger RNA or ribosomal RNA.

8. The method according to claim 1, wherein step (i) comprises immobilizing cells contained within the biological sample onto a solid support followed by contacting the cells with a fixation and/or permeabilisation reagent.

9. The method according to claim 1, wherein step (ii) comprises fluorescence microscopy in combination with digital image acquisition by charge coupled device.

10. The method according claim 1, wherein step (iv) is performed manually using image-analysis software.

11. The method according to claim 1, wherein step (iv) is performed in an automated fashion.

12. The method according to claim 1, further comprising the step of providing a clinical diagnosis, comprising determining the relative abundance of CWDB-positive cells, and correlating the relative abundance with the presence of a CWDB-related disease or condition.

13. The method according to claim 12, comprising analyzing at least 50 white blood cells and wherein an amount of at least 3 CWDB-positive white blood cells per 50 white blood cells is indicative of a CWDB-related disease or condition.

14. The method according to claim 12, wherein said CWDB is Coxiella burnetii and wherein said CWDB-related disease or condition is Q-fever.

15. The method according to claim 12, wherein said CWDB is Borrelia burgdorferi and wherein said CWDB-related disease or condition is Lyme's-disease.
